(19) United States
(12) Patent Application Publication
(10) Pub. No.: US 2010/0310488 A1
(43) Pub. Date: Dec. 9, 2010

(54) SELECTING/IDENTIFYING AND ADMINISTERING ACTIVE AGENTS USEFUL FOR MODIFYING THE SHAPE OF KERATIN/HAIR FIBERS

(75) Inventors: Sylvain MALGOURIES, Paris (FR); Sébastien THIBAUT, Puteaux (FR)

Correspondence Address: BUCHANAN, INGERSOLL & ROONEY PC, POST OFFICE BOX 1404, ALEXANDRIA, VA, 22313-1404, US

(73) Assignee: L'OREAL, Paris (FR)

(21) Appl. No.: 12/796,839

(22) Filed: Jun. 9, 2010

(30) Foreign Application Priority Data
Dec. 10, 2007 (EP) PCT/EP2008/067240
Dec. 10, 2007 (FR) 0759675

(51) Int. Cl. C12Q 1/02; A61Q 5/04; A61K 8/46

(52) U.S. Cl. 424/70.5; 435/29; 424/70.2

(57) ABSTRACT

A method of selecting/identifying an active agent for modifying the shape of keratin fibers, includes the following steps:
a) culturing mammalian cells,
b) adding a test substance to the mammalian cell culture,
c) after incubation thereof, assaying the amount of glycosaminoglycans (GAGs) in the mammalian cells or in the mammalian cell culture medium,
d) comparing the amount of GAGs measured in step c) with that measured starting from a control culture of mammalian cells, obtained under the same conditions, but in the absence of test substance, and selecting the substances for which the amount of GAGs is modified by at least a factor of 0.5 relative to the amount measured in the control culture; the active agents thus selected/identified are useful for modifying the shape of keratin fibers.

### CROSS-REFERENCE TO PRIORITY/PCT APPLICATIONS

This application claims priority under 35 U.S.C. §119 of FR 0759675, filed Dec. 10, 2007, and under 35 U.S.C. §120 of U.S. Provisional Application No. 61/014,910, filed Dec. 19, 2007, and is a continuation of PCT/EP 2008/067240, filed Dec. 10, 2008 and designating the United States (published in the English language on Jun. 18, 2009 as WO 2009/074613 A1), each hereby expressly incorporated by reference in its entirety and each assigned to the assignee hereof.

### BACKGROUND OF THE INVENTION

1. Technical Field of the Invention

The present invention relates to a cosmetic treatment for keratin fibers and/or for their support, such as hairy skin, in particular for the hair and/or the scalp, with a view to modifying the shape of the keratin fibers in a long-lasting manner, more specifically for straightening them and smoothing them out.

2. Description of Background and/or Related and/or Prior Art

It is known that two techniques are conventionally employed for permanently reshaping the hair. They are based on cleavage of the disulfide bonds present in keratin (cystine):
the first comprises, in a first stage, opening the disulfide bonds using a composition containing a reducing agent, and then, after having preferably rinsed the hair, reconstituting said disulfide bonds in a second stage, by applying to the hair, which has been placed under tension beforehand with rollers or the like or shaped or smoothed out by other means, an oxidizing composition also known as a fixer, so as to give the head of hair the desired shape—this technique equally makes it possible either to make the hair wavy or to straighten it, uncurl it or smooth it out;
the second comprises conducting a “lanthionization” operation using a composition containing a base belonging to the hydroxide family—this leads to a replacement of the disulfide bonds (—CH₂—S—S—CH₂—) with lanthionine bonds (—CH₂—S—CH₂—) and this technique does not require a fixing reaction and is therefore carried out in a single stage.

These techniques give good results and the new shape provided the hair is long-lasting and withstands, in particular, the action of washing with water or with shampoos. However, they involve the use of substances of which the odor may be deemed unpleasant by the user and may be tricky to implement. In certain cases, these treatments may result in the hair becoming brittle.

Need therefore still exists to develop new methods which are suitable for the cosmetics field, which are not very aggressive for the hair and the scalp and which make it possible to modify the natural shape of said hair in a long-lasting manner.

Glycosaminoglycans (GAGs) are anionic polysaccharides grafted onto protein cores. GAGs group together chondroitin sulfates (CS), heparan sulfates (HS), keratan sulfates (KS) and dermatan sulfates (DS) according to their chemical structure.

Glycosylated proteins have for a long time been considered to be structural compounds of the extracellular matrix. Their involvement in many functions, such as cell communication, proliferation or differentiation, has only recently been demonstrated.

### SUMMARY OF THE INVENTION

Unexpectedly, it has now been found that in individuals with curly hair, the distribution of glycosaminoglycans (or GAGs) in the bulb of the hair follicle is asymmetrical.

It, therefore, has now been found that it is possible to modify the shape of the keratin fiber, in particular newly synthesized by the hair follicle, by acting on the distribution of the GAGs, in particular by modulating the amount thereof at the level of the hair follicle. Indeed, increasing the synthesis of GAGs in the follicle of the curly hair will have the effect of making the distribution thereof uniform and therefore of making the head hair or body hair taut and/or straightening it.

Likewise, agents which inhibit the synthesis of GAGs in follicles, by accentuating this distribution asymmetry, will increase the curliness or the curving of the head hair, of the body hair or of the eyelash.

The present invention therefore features the use of compounds which modulate the synthesis or degradation of GAGs, for modulating the shape of keratin fibers, in particular newly synthesized by the hair follicle.

This invention in particular features the use of compounds which stimulate the synthesis and/or secretion of proteoglycans (PGs) and/or of GAGs, and/or which decrease the degradation of GAGs, as an agent for modulating the shape of keratin fibers, in particular for straightening them or smoothing them out.

According to another aspect, the present invention features the use of compounds which decrease or inhibit the synthesis and/or secretion of PGs and/or of GAGs, and/or which stimulate the degradation of GAGs, as an agent for modulating the shape of keratin fibers, in particular for increasing, promoting or maintaining the curly and/or frizzy appearance of keratin fibers, or promoting waviness, and/or increasing the curve of keratin fibers.

This invention also features a cosmetic treatment regime or regimen for keratin fibers in a mammal, for modifying the shape thereof, comprising the application to the keratin fibers or to their support of at least one compound which modulates the synthesis and/or secretion of proteoglycans (PGs) and/or of glycosaminoglycans and/or which modulates the degradation of GAGs as described above.

The process and the use according to the invention are particularly suitable for modulating/modifying the shape of the newly synthesized fiber.

This constitutes an advantage of the invention according to which, rather than straightening a fiber which has grown curly, it transpires that the fiber grows straight from its root onwards.

Compositions suitable for the implementation thereof are also provided by the present invention.

According to the invention, the term “keratin fibers” means fibers of human or animal origin, such as the hair, body hairs, eyelashes, wool, angora, cashmere or fur. Although the invention is not limited to particular keratin fibers, reference will nevertheless more particularly be made to the hair and to the eyelashes.

WO 99/24009 proposes the administration of D-xylose or of certain esters thereof for improving the functionality of epidermal cells, by stimulating the synthesis of PGs and of GAGs.

EP-531111 relates to compositions for promoting hair growth, containing a monosaccharide derivative which is a glycosaminoglycanase inhibitor.

EP-0277428 relates to the administration of inhibitors of proteoglycanase and glycosaminoglycanases for improving hair growth.

EP-0398669 relates to sugar lactone glucuronide derivatives for improving hair growth.

EP-0348184 relates to glycosaminoglycanase inhibitors of the aldonomonolactone, alduromonolactone and acetylated monosaccharide type for improving hair growth.

EP-211610 and EP-354595 describe the use of oligosaccharides, in particular of disaccharides comprising a uronic acid residue, for promoting hair growth.

WO 02/051828 describes novel C-glycoside derivatives and the use thereof, in particular for stimulating GAG synthesis. It is in particular proposed to incorporate them into an anti-hair-loss lotion or gel.

WO 2006/090307 reports the use of C-glycoside derivatives for improving the mechanical strength of keratin fibers, and in particular for preventing breaking of the hair.

However, to the knowledge of the assignee hereof, it has never been recognized to use compounds which stimulate the synthesis of GAGs and/or PGs, or which decrease the degradation of GAGs, for straightening and/or smoothing out the hair, body hair and/or the eyelashes. Conversely, it has never been recognized to use compounds which decrease or inhibit the synthesis and/or secretion of PGs and/or of GAGs, and/or which stimulate the degradation of GAGs, for making keratin fibers wavy or curling them. The terms “straightening”, “smoothing out”, “making wavy” or “curling” are herein intended to mean the modification of the shape of the fiber, and more especially during the production of the latter by the hair follicle.

### BRIEF DESCRIPTION OF THE DRAWINGS

FIGS. 1A and 1B depict the analysis of immunolabeled dissected hair follicles, and

FIG. 2 presents the results of assaying of sulfated glycosaminoglycans according to the present invention.

### DETAILED DESCRIPTION OF BEST MODE AND SPECIFIC/PREFERRED EMBODIMENTS OF THE INVENTION

Indeed, it has now been demonstrated that the distribution of GAGs in the hair follicle is not uniform, and that the expression of some of them greatly decreases under the cells of the matrix, i.e., under the proliferative compartment of the follicle. This is, in particular, the case for perlecan and the heparan sulfate chains, which are greatly under-represented in the basal membrane. Similarly, nonsulfated and 4-sulfated chondroitins, D-type chondroitins and keratan sulfates are greatly decreased in the connective sheath. This decrease in expression could be correlated with the proliferative capacity of the matrix cells.

In addition, surprisingly, it has now been demonstrated that the distribution of these glycosaminoglycans is asymmetrical, in the bulb of a curly hair, or in the follicle of a curved eyelash. This asymmetry concerns in particular the following compounds: perlecan, heparan sulfates, nonsulfated and 4-sulfated chondroitins, D-type chondroitins, and keratan sulfates. The decrease in GAG expression reported above is greater on the convex side of the curvature of the follicle.

The use of modulators of the synthesis and/or degradation of GAGs, in particular of an inhibitor of degradation, makes it possible, by making the synthesis of GAGs in the bulb of the hair, in particular of the curly hair, uniform, to even out their distribution. The use of activators of the synthesis and/or inhibitors of the degradation of GAGs according to the invention will thus result in the hair becoming taut, and in the case of frizzy or curly hair, to the hair being smoothed out and/or straightened.

A novel approach for modifying the shape of keratin fibers, in particular the hair or the eyelash, has thus been found.

For this reason, the present invention features a method of selecting/identifying an active agent for modifying the shape of keratin fibers, which comprises the following steps:
a) culturing mammalian cells,
b) adding the test substance to the mammalian cell culture,
c) after incubation thereof, assaying the amount of glycosaminoglycans (GAGs) in the mammalian cells or in the mammalian cell culture medium,
d) comparing the amount of GAGs measured in step c) with that measured starting from a control culture of mammalian cells, obtained under the same conditions, but in the absence of test substance, and
e) selecting the substances for which the amount of GAGs is modified by at least a factor of 0.5 relative to the amount measured in the control culture.

The mammalian cells that can be used for carrying out the technique according to the invention may be selected by one skilled in the art from skin cells and/or hair follicle cells, or else from immortalized lines such as HEK, CHO, HaCat, 3T3, etc. Fibroblasts or keratinocytes will in particular be employed. Advantageously, the fibroblasts are selected from the fibroblasts of the connective sheath of the hair, the fibroblasts of the dermal papilla and the fibroblasts of human dermis.

In particular, the method of selecting/identifying an active agent useful for modifying the shape of keratin fibers according to the invention will comprise the following steps:
a) culturing fibroblasts,
b) adding the test substance to the fibroblast culture,
c) after incubation thereof, assaying the amount of glycosaminoglycans (GAGs) in the fibroblasts or in the fibroblast culture medium,
d) comparing the amount of GAGs measured in step c with that measured starting from a control culture of fibroblasts, obtained under the same conditions, but in the absence of test substance, and
e) selecting the substances for which the amount of GAGs is modified by at least a factor of 0.5 relative to the amount measured in the control culture.

The steps for culturing the mammalian cells, in particular fibroblasts or keratinocytes, will be carried out by techniques and in media known to those skilled in the art.

In step c), the test substance will be maintained in contact with the fibroblasts or the keratinocytes for a period of time sufficient for the GAG metabolism to be influenced, where appropriate, by the presence of said substance. This period of time is from a few hours to a few days, especially from 6 h to 15 days, in particular from 1 to 6 days.

Similarly, the assaying of the GAGs will be carried out by methods known to those skilled in the art, such as the use of cationic dyes, for instance 1,9-dimethylmethylene blue (DMMB) (Barbosa, I. et al., Improved and simple micro assay for sulfated glycosaminoglycans quantification in biological extracts and its use in skin and muscle tissue studies, Glycobiology, 2003, 13, 647-653) or O-safranin (Lammi, M. et al., Densitometric assay of nanogram quantities of proteoglycans precipitated on nitrocellulose membrane with Safranin O, Anal Biochem, 1988, 168, 352-357), or by assaying uronic acid via the carbazole reaction (Dische, Z. et al., Two modifications of the carbazole reaction of hexuronic acids for the differentiation of polyuronides, Anal Biochem, 1967, 21, 125-130).

Substances capable of modulating the GAG concentration in the hair follicle, and therefore of modifying the shape of the hair, body hair and/or eyelashes in a long-lasting manner, will thus be selected. After the cells have been cultured in the presence of the compounds or mixtures of compounds selected, the GAG concentration may in fact increase by 1.5 times, or, on the contrary, be less than or equal to 0.5 times the concentration measured in the control culture.

According to one of the embodiments of the method of the invention, in step e), the substances for which the amount of GAGs is at least equal to 1.5 times the amount measured in the control culture are selected.

According to another embodiment of the method according to the invention, in step e), the substances for which the amount of GAGs is decreased at least by half relative to the amount measured in the control culture are selected.

This invention thus features the cosmetic application, in a composition containing a physiologically acceptable medium, as an active agent for modifying the shape of keratin fibers, of at least one modulator of the synthesis and/or secretion and/or degradation of GAGs, this active agent being selected by the technique described above, the modulator or the composition containing it being useful for modifying the shape of keratin fibers.

In particular, the present invention features the cosmetic application, in a composition containing a physiologically acceptable medium, as an active agent for modifying the shape of keratin fibers, of at least one active agent selected from agents which stimulate the synthesis and/or secretion of GAGs and of PGs and agents which decrease the degradation of GAGs.

The cosmetic regime or regimen according to the invention is more particularly suitable for keratin fibers which are human hair, human eyelashes and/or human body hair.

More particularly, the active agent which stimulates the synthesis and/or secretion of GAGs and of PGs and/or the active agent which decreases the degradation of GAGs, or the composition containing it, is useful for smoothing out and/or straightening the hair and/or the eyelashes.

The term “straightening” encompasses, according to the invention, the straightening, smoothing out or uncurling of Caucasian, Asian, North African or African hair.

According to another embodiment, this invention features the cosmetic application, in a composition containing a physiologically acceptable medium, as an active agent for modifying the shape of keratin fibers, of at least one active agent selected from among agents which decrease or inhibit the synthesis and/or secretion of PGs and/or GAGs, and/or which stimulate the degradation of GAGs.

More particularly, the active agent which decreases or inhibits the synthesis and/or secretion of PGs and/or of GAGs, and/or which stimulates the degradation of GAGs, is administered as an agent for increasing the curve of keratin fibers, in particular newly synthesized keratin fibers.

The hair shaping according to the invention encompasses the curling, permanent-waving and setting of hair, in particular Caucasian, Asian, North African, Afro-American or African hair. The active agents may also be administered according to the invention for promoting the curved shape of eyelashes, by increasing and/or maintaining the natural curve.

One aspect of the invention is also the cosmetic application of at least one activator of the synthesis and/or secretion of GAGs and/or of an inhibitor of the degradation of GAGs as described above, as an active agent for straightening and/or smoothing out the hair. In particular, it features the formulation of at least one activator of the synthesis and/or secretion of GAGs and/or of an inhibitor of the degradation of GAGs into a composition useful to be ingested or applied to the skin, the mucous membranes or the keratin fibers, as an active agent for straightening and/or smoothing out the hair.

The agents that are of useful for implementing the invention, for modifying the shape of keratin fibers, are preferably modulators of the synthesis and/or secretion and/or degradation of GAGs, and are in particular selected from competitors of GAG synthesis, sugars which are GAG precursors, modulators of the enzymes which initiate GAG synthesis, modulators of the enzymes responsible for GAG synthesis elongation, modulators of the enzymes responsible for post-synthetic modifications of GAGs, heparanase 1 or 2 modulators, and glycanase modulators.

Such compounds are known to those skilled in the art.

Exemplary are:

Competitors of glycosaminoglycan synthesis, such as: 2-acetamido-1,3,6-tri-O-acetyl-2,4-dideoxy-D-xylohexopyranose (Hull, R. L. et al., Inhibition of glycosaminoglycan synthesis and protein glycosylation with WAS-406 and azaserine result in reduced islet amyloid formation in vitro, Am J Physiol Cell Physiol, 2007, 293, C1586-C1593);

Sugars which are precursors of glycosaminoglycans, products obtained from or mimetic compounds, in particular D-xylose and D-galactose, esters thereof and the oligosaccharides containing them (Fritz, T. A. et al., Biosynthesis of heparan sulfate on beta-D-xylosides depends on aglycone structure, J Biol Chem, 1994, 269, 300-307; Robinson, H. C. et al., The effect of D-xylose, beta-D-xylosides and beta-D-galactosides on chondroitin sulfate biosynthesis in embryonic chicken cartilage, Biochem J, 1975, 148, 25-34), and particularly those capable of increasing glycosaminoglycan synthesis;

Plant extracts capable of modulating the amount and/or the quality of glycosaminoglycans, in particular extracts of plants of the Filicium family (WO 1997001346 A1) and the Foetida family (WO 1997001345 A1);

Agents which modulate the following enzymes, involved in the initiation of glycosaminoglycan synthesis:

xylosyltransferase 1 (XT1), xylosyltransferase 2 (XT2), beta-1,4-galactosyltransferase 1 to 7, beta-1,3-galactosyltransferase 1 to 8, galactosyltransferase I, galactosyltransferase II, N-acetylglucosaminyltransferase, glucuronyltransferase, beta-1,3-glucuronyltransferase 3 (glucuronosyltransferase I), beta-1,3-glucuronyltransferase 2 (glucuronosyltransferase S), beta-1,3-glucuronyltransferase 1 (glucuronosyltransferase P), UDP-GaINAc N-acetylgalactosaminyltransferase 1 to 15, beta 1,3-N-acetylgalactosaminyltransferase 2, beta-1,3-N-acetylglucosaminyltransferase 1 to 8, alpha-1,4-N-acetylglucosaminyltransferase;

Agents which modulate the following enzymes, involved in glycosaminoglycan synthesis elongation:

heparan sulfate GlcA/GlcNAc transferase 1 (exostosin-1, EXT1); heparan sulfate GlcA/GlcNAc transferase 2 (exostosin-2, EXT 2); α-4-N-acetylhexosaminyltransferase (exostosin-like 2, EXTL2); heparan sulfate GlcA/GlcNAc transferase like 1 (exostosin-like 1, EXTL1); heparan sulfate GlcA/GlcNAc transferase like 3 (exostosin-like-3, EXTL3); N-acetylglucosaminyltransferase; heparan sulfate synthase; chondroitin sulfate synthase 1/chondroitin synthase; chondroitin sulfate synthase 2/chondroitin polymerizing factor; chondroitin sulfate synthase 3; chondroitin sulfate glucuronyltransferase; chondroitin sulfate N-acetylgalactosaminyltransferase 1; chondroitin sulfate N-acetylgalactosaminyltransferase 2; hyaluronan synthases 1 to 3;

Agents which modulate the following enzymes, involved in post-synthetic modifications of glycosaminoglycans:

N-deacetylase/N-sulfotransferase, C-5 epimerase, 2-O sulfotransferase, 6-O sulfotransferase, 3-O sulfotransferase, N-deacetylase/N-sulfotransferase-1 to 4, heparan sulfate 6-sulfotransferase-1 to 3, glucosaminyl 3-O-sulfotransferase-1,2,3A, 3B and 4, heparan sulfate 2-sulfotransferase, heparan sulfate 3-OST-5, chondroitin D-N-acetylgalactosamine-4-O-sulfotransferase 1 to 3, dermatan-4-sulfotransferase-1, N-acetylgalactosamine-4-O-sulfotransferase 1 and 2, chondroitin 6-sulfotransferase 1 and 2, keratan sulfate D-galactose-6-O sulfotransferase, corneal N-acetylglucosamine-6-sulfotransferase, carbohydrate N-acetylglucosamine-6-O-sulfotransferase 2, intestinal N-acetylglucosamine 6-O-sulfotransferase, dermatan/chondroitin sulfate 2-O-sulfotransferase, N-acetylgalactosamine 4-sulfate 6-O-sulfotransferase, GalNAc (4SO4)6-O-sulfotransferase, uronosyl 2-O-sulfotransferase, HEC-GlcNAc 6-O-sulfotransferase;

Agents which modulate the expression or activity of the heparanase-1 and heparanase-2 degradation enzymes;

Agents which modulate glycanases (involved in the degradation of glycosaminoglycans):
iduronate-2-sulfatase, alpha-L-iduronidase, heparan sulfatase, N-acetylglucosamine-6-sulfatase, N-sulfoglucosamine sulfohydrolase (heparan N-sulfatase, sulfamidase), galactosamine (N-acetyl)-6-sulfate sulfatase, N-acetylgalactosamine-4-sulfatase (arylsulfatase B), alpha-N-acetylglucosaminidase(N-acetylglucosulfatase), acetyl CoA:alpha-glucosaminide acetyltransferase (glucosamine-N-acetyltransferase), N-acetylglucosamine 6-sulfatase, beta-glucuronidase, Beta-glucuronidase-like 3,4,5 (SMA3,4,5), LOC 153561, beta-galactosidase, N-alpha-acetylglucosaminidase, N-acetyl-alpha-D-glucosaminidase, galactosamine-6-sulfate sulfatase, galactosamine-6-sulfatase, N-acetylgalactosamine 4-sulfatase, beta-D-glucuronidase, glucosamine-6-sulfate sulfatase, hyaluronidase, beta-glucuronidase, beta-N-acetylhexosaminidase A or B, beta-glucuronidase.

In particular, useful are those active agents which inhibit the synthetic and elongation enzymes indicated above:

Analogues of 4-methylumbelliferone, and of 1-deoxy-GlcNAc, such as 2-acetamido-3,4,6-tri-O-acetyl-1,5-anhydro-2-deoxy-D-glucitol;

Uridine derivatives, such as 5′-(6-O-[10-(2-naphthyl)-3,6,9-trioxadecanyl]-alpha-d-galactopyranosyl)diphosphate (beta-1,4-galactosyltransferase inhibitor).

These compounds are preferably formulated into compositions for increasing the curve of the eyelash and/or for promoting or maintaining the curly and/or frizzy appearance of the hair, or for promoting the waviness of the hair.

Other compounds that are useful according to the invention are inhibitors of the degradation enzymes indicated above:

Derivatives of iduronosyl anhydromannitol 6-sulfate (IMs), iduronosyl anhydromannitol, phenyl iduronide (Phl) or of 4-methylumbelliferyl iduronide (alpha-L-iduronidase inhibitors);

S-, N- and O-glycosyl derivatives of the compound 2-acetamido-2-deoxy-D-glucose, or derivatives of 4-hydroxy-2-imino-1-methylpyrrolidine-5-carboxylic acid and 2-imino-1-methylpyrrolidine-5-carboxylic acid from streptomyces (alpha-N-acetylglucosaminidase inhibitors);

Derivatives of aldonomonolactone or of alduronomonolactone, and acylated monosaccharides (glycosaminoglycanase inhibitors), and more generally sugar lactone derivatives. Such compounds are indicated in EP-0384184, EP-0277428, EP-0398669 and EP-531111.

These compounds or the compositions containing them are preferably for straightening and/or smoothing out the hair.

Advantageously, the modulator of the synthesis and/or secretion and/or degradation of GAGs according to the invention is applied in the form of a composition containing a physiologically acceptable medium, i.e., a medium compatible with the skin and/or the mucous membranes. The compositions according to the invention are in particular cosmetic or dermatological compositions; they are in particular a composition suitable for caring for, conditioning, making up, removing makeup from, protecting, cleansing or washing keratin fibers.

For oral administration, the composition of the invention may be in any of the suitable forms, particularly in the form of an oral solution, a tablet, a gel capsule, a capsule or else a nutritional food or a nutritional supplement.

Said composition also comprises at least one appropriate excipient suitable for oral administration.

In these compositions, the concentration of the modulator of the synthesis and/or secretion and/or degradation of GAGs may range from 10⁻³ to 30% by weight; it is in general greater than or equal to 0.01% by weight, relative to the total weight of the composition, in particular greater than or equal to 0.1%; the effective amounts will be adjusted by one skilled in the art according to the desired result and the modulators of the synthesis and/or secretion and/or degradation of GAGs used, but are generally less than or equal to 30%, in particular less than or equal to 15% by weight, or even less than or equal to 10%.

The subject compositions may be in any of the forms suitable for caring for the hair and/or the scalp, in particular in the form of a haircare lotion, for example for daily or twice-weekly application, a shampoo or a hair conditioner, in particular for weekly or twice-weekly application, a liquid or solid soap for cleansing the scalp, for daily application, a product for shaping the hairstyle (lacquer, hairstyling gel), a treatment mask, or a foaming gel or cream for cleansing the hair.

A composition suitable for carrying out the invention may be in the form of an aqueous, alcoholic or aqueous-alcoholic solution or suspension, an oily suspension, an emulsion of more or less fluid and in particular liquid, semi-liquid or solid consistency, obtained by dispersion of a fatty phase in an aqueous phase (O/W) or vice versa (W/O), or a multiple emulsion, an aqueous, aqueous-alcoholic or oily gel, a loose or compacted powder to be used as it is or to be incorporated into a physiologically acceptable medium, microcapsules or microparticles, or ionic or nonionic dispersions.

Advantageously, the modulator of the synthesis and/or secretion and/or degradation of GAGs is topically applied in the form of a formulation which promotes the penetration of the agent into the hair follicle.

The compositions may, in certain cases, contain penetration accelerators, in particular selected from among noncyclic monoalcohols and dialcohols, ethyl acetate, butyl acetate, isopropyl myristate, fatty acids, phospholipids, terpenes, azone and derivatives, propylene glycol and glycolic derivatives, cyclodextrins, octyl salicylate, cyclopentadecanolid, polysorbates, and polyvinylpyrrolidone and derivatives, this list not being limiting.

The modulator(s) of the synthesis and/or secretion and/or degradation of GAGs may in particular be in a formulation which includes individualized porous particles characterized in that they have a volume-average diameter of less than or equal to 10 μm and a specific surface area of greater than or equal to 1 m²/g, as described in FR-2856594.

According to one embodiment, the compositions also contain a surfactant of nonionic, anionic, cationic or amphoteric type, and among the latter, exemplary are alkyl sulfates, alkyl benzenesulfates, alkyl ether sulfates, alkyl sulfonates, quaternary ammonium salts, alkylbetains, oxyethylenated alkylphenols, fatty acid alkanolamides, oxyethylenated fatty acid esters, and also other nonionic surfactants of the hydroxypropyl ether type.

When the compositions contain at least one surfactant, the latter is generally present at a maximum concentration of 30% by weight, and preferably from 0.5% to 10% by weight, relative to the total weight of the composition.

With the goal of improving the cosmetic properties of the hair or alternatively of reducing or preventing the degradation thereof, the composition may also contain a treatment agent of cationic, anionic, nonionic or amphoteric nature.

Among the treatment agents which are particularly preferred, especially exemplary are those described in FR-2598613 and FR-2470596. As treatment agents, exemplary are linear or cyclic, volatile or non-volatile silicones and mixtures thereof, polydimethylsiloxanes, quaternized polyorganosiloxanes such as those described in FR-2535730, polyorganosiloxanes comprising aminoalkyl groups modified with alkoxycarbonylalkyl groups, such as those described in U.S. Pat. No. 4,749,732, polyorganosiloxanes such as the polydimethylsiloxane-polyoxyalkyl copolymer of the Dimethicone Copolyol type, a polydimethylsiloxane containing stearoxy (stearoxydimethicone) end groups, a polydimethylsiloxane-dialkylammonium acetate copolymer or a polydimethylsiloxane-polyalkylbetain copolymer described in GB-2197352, polysiloxanes organomodified with mercapto or mercaptoalkyl groups, such as those described in FR-1530369 and in EP-295780, and also silanes such as stearoxytrimethylsilane.

The compositions according to the invention may also contain other treatment ingredients such as cationic polymers, for instance those included in the compositions of FR-7932078 (2.472.382) and FR-8026421 (2.495.931), or else cationic polymers of the ionene type, such as those included in the compositions of LU-83703, basic amino acids (such as lysine or arginine) or acidic amino acids (such as glutamic acid or aspartic acid), peptides and derivatives thereof, protein hydrolysates, waxes, swelling agents and penetrating agents such as the SiO₂/PDMS (polydimethylsiloxane) mixture, dimethylisosorbitol, urea and its derivatives, pyrrolidone, N-alkylpyrrolidones, thiamorpholinone, alkylene glycol alkyl ethers or dialkylene glycol alkyl ethers, for instance propylene glycol monomethyl ether, dipropylene glycol monomethyl ether, ethylene glycol monoethyl ether and diethylene glycol monoethyl ether, 2-imidazolidinone, and also other compounds such as fatty alcohols, lanolin derivatives, active agents such as panthothenic acid, agents for preventing hair loss, antidandruff agents, thickeners, suspension agents, sequestering or complexing agents, opacifiers, sunscreens and also fragrances and preservatives.

The compositions according to the invention are in particular in the form of a thickened cream so as to keep the hair as stiff as possible. These creams are prepared in the form of “heavy” emulsions, for example based on glyceryl stearate, on glycol stearate, on self-emulsifiable waxes or on fatty alcohols.

Also useful are liquids or gels containing thickeners, such as carboxyvinyl polymers or copolymers which “stick” the hairs together and keep them in the smooth position during the leave-in time.

The compositions according to the invention may also contain at least one adjuvant selected from among silicones in soluble, dispersed or microdispersed form, nonionic, anionic, cationic and amphoteric surfactants, ceramides, glycoceramides and pseudoceramides, vitamins and provitamins, including panthenol, plant, animal, mineral and synthetic oils, waxes other than ceramides, glycoceramides and pseudoceramides, silicone-based or nonsilicone-based, water-soluble and fat-soluble sunscreens, pearlescent agents and opacifiers, sequestering agents, plasticizers, solubilizing agents, acidifying agents, mineral and organic thickeners, antioxidants, hydroxy acids, penetration agents, fragrances and preservatives. Among the solubilizing agents, exemplary are lower alcohols such as, for example, ethanol, propanol or isopropanol.

The compositions according to the invention contain at least one, and in particular at least two, modulators of the synthesis and/or secretion and/or degradation of GAGs.

However, an active agent which increases the concentration of GAGs will preferably not be combined with an active agent which decreases this same concentration.

According to one particular embodiment, the subject compositions also contain at least one other active agent that is beneficial to the health and vigor of the hair and of body hair, in particular at least one active agent which promotes hair regrowth and/or which limits hair loss. It may in particular be an inhibitor of the 15-hydroxyprostaglandin dehydrogenase enzyme, such as those described in WO 2004/073594, or esters of vitamin F and of glucose described in EP-1371658.

This invention also features a cosmetic treatment regime or regimen for keratin fibers in a mammal, for modifying their shape, wherein at least one modulator of the synthesis and/or secretion and/or degradation of GAGs, or a composition containing same, as described above, is administered to the mammal. The mammal is preferably a human being.

According to one embodiment, the modulator of the synthesis and/or secretion and/or degradation of GAGs, or the composition containing it, is topically applied to the keratin fibers or to their support, in particular to the hair and/or the scalp.

The regime or regimen according to the invention may, for example, be carried out by applying the modulator(s) of the synthesis and/or secretion and/or degradation of GAGs or the compositions containing them, as described above, to the hair and/or the scalp, daily or twice a day, for a period preferably of at least one week, and in particular from 2 to 6 weeks. The modulator of the synthesis and/or secretion and/or degradation of GAGs or the composition may be rinsed off after a sufficient contact time, in particular from 5 to 60 minutes, or else may be applied, for example, in the form of a leave-in lotion or nourishing cream and remain in contact with the hair and/or the scalp until the next shampooing operation. The process according to the invention makes it possible to provide a long-lasting modification of the shape of the hair, since it acts on the structure of the hair as soon as the latter is formed. It will be particularly suitable for individuals who have naturally curly or frizzy hair; in one particular embodiment, the hair will be placed under tension or shaped or smoothed out by appropriate means.

According to another embodiment, the modulator of the synthesis and/or secretion and/or degradation of GAGs or the composition containing it will be administered to the mammal orally.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Demonstration of the Asymmetrical Distribution of GAGS on Curly Hair

1.a) Dissection of Hair Follicles:

Hair follicles originating from human scalp biopsies are dissected according to the method described in FR-2736721A1 of 17/01/97; U.S. Pat. No. 5,712,169 of 28/01/98.

The isolated follicles are conserved in Tissue-Tek OCT compound (Miles, Naperville, Ill., USA) and frozen in a dry ice/ethanol mixture.

1. b) Preparation of Cryosections:

Longitudinal 7 μm-thick sections of isolated hair follicles or scalp biopsies are cut using a CM3050 cryostat (Leica, Rueil Malmaison, France), in which the object temperature is maintained at −40° C. and the chamber temperature is maintained at −35° C. The sections are fixed in air and then conserved for 24 hours at +4° C.

1. c) Immunolabeling:

The sections are fixed with acetone at −20° C. for 10 minutes and then rinsed in a phosphate-buffered saline (PBS) (Sigma, Saint-Quentin Fallavier, France). The endogenous peroxidases are saturated using 0.1% hydrogen peroxide (Sigma, Saint-Quentin Fallavier, France).

List of the Antibodies Used:

| Primary | | | | |
|---|---|---|---|---|
| antibody | Ig class | Specificity | Dilution | Supplier |
| F58-10E4 | Mouse IgM | heparan sulfate | 1/50 | Seikagaku corporation, |
| | | | | Tokyo, Japan |
| HepSS-1 | Mouse IgM | heparan sulfate | 1/30 | Seikagaku corporation, |
| | | | 1/100 | Tokyo, Japan |
| 1-B-5 | Mouse IgG1 | Unsulfated | 1/50 | Seikagaku corporation, |
| | | chondroitin or | | Tokyo, Japan |
| | | Dermatan | | |
| | | (chABC) | | |
| 2-B-6 | Mouse IgG1 | 4-sulfated | 1/20 | ICN Biomedicals |
| | | chondroitin | | |
| | | (chABC) | | |
| BE-123 | Mouse IgG1 | 4-sulfated | 1/20 | MP Biomedicals, Aurora, |
| | | chondroitin | | OH, USA |
| | | (chABC) | | |
| 3-B-3/C1 | Mouse IgM | 6-sulfated | 1/20 | ICN Biomedicals, Costa |
| | | chondroitin | | Mesa, CA, USA |
| | | (chABC) | | |
| MK-302 | Mouse IgG1 | 6-sulfated | 1/20 | MP Biomedicals, Aurora, |
| | | chondroitin | | OH, USA |
| | | (chABC) | | |
| MO-225 | Mouse IgM | Chondroitin- | 1/50 | Seikagaku corporation, |
| | | sulfate type D | | Tokyo, Japan |
| 5-D-4 | Mouse IgG1 | Keratan-sulfate | 1/50 | Seikagaku corporation, |
| | | | | Tokyo, Japan |
| 7B5 | Mouse IgG1 | Perlecan | 1/100 | Zymed, South San |
| | | | | Francisco, CA, USA |

| Secondary | | | | | |
|---|---|---|---|---|---|
| antibody | Conjugate | Specificity | Dilution | Host | Supplier |
| 705-065-147 | Biotin | Goat | 1/400 | Donkey | Jackson, Baltimore, |
| | | | | | PE, USA |
| E0433 | Biotin | Mouse IgG | 1/400 | Goat | DAKO, Glostrup, |
| | | | | | Denmark |
| E432 | Biotin | Rabbit | 1/400 | Goat | DAKO, Glostrup, |
| | | | | | Denmark |
| A31570 | Alexafluor | Mouse IgG | 1/100 | Donkey | Molecular Probes, |
| | 555 | | | | Eugene, OR, USA |
| A11055 | Alexafluor | Goat | 1/100 | Donkey | Molecular Probes, |
| | 488 | | | | Eugene, OR, USA |

The labeling is then analyzed with a Zeiss Axioscope microscope (Carl Zeiss, Oberkochen, Germany).

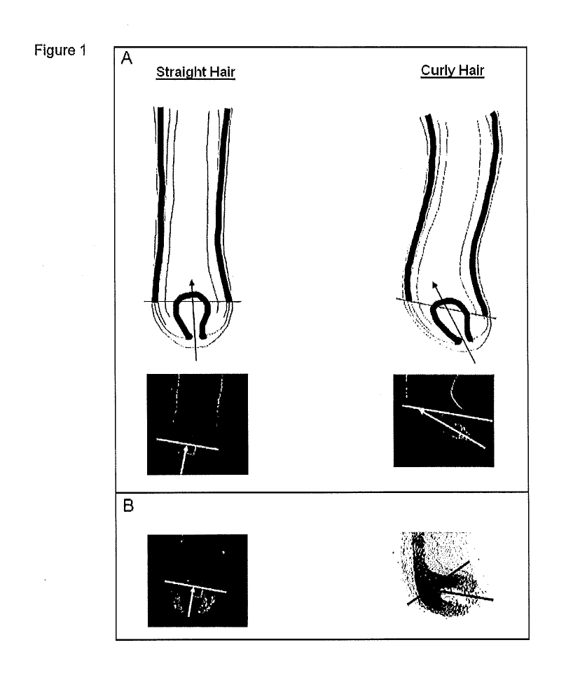

The results are represented in FIG. 1.

An asymmetrical distribution of the heparan sulfate chains is observed in the bulb of a frizzy hair, compared with a straight hair. FIG. 1B shows the asymmetrical proliferation which results therefrom.

### Example 2

### Increase in GAG Synthesis with a Xyloside Derivative

Fibroblast Culture:

The fibroblasts are cultured in DMEM medium, 10% foetal calf serum (Gibco, Invitrogen), 1 mM MEM pyruvate (Gibco, Invitrogen) and 25 units/ml of penicillin-streptomycin (Gibco, Invitrogen). The dermal papilla fibroblast (P6), connective sheath fibroblast (P6) and dermal fibroblast are then counted using a Coulter Counter Z2 (Beckman Coulter inc., Fullerton, USA) and seeded at 2×10⁵ cells per well in a 12-well plate, with the wells containing 1.5 ml of medium alone, which is the “control” condition, or being treated with 3 mM of 1,5-anhydro-6,8-dideoxy-L-glucooctitol. After 7 days, the supernatant is recovered and stored at −80° C. until the GAGs are assayed. The cells are lysed with 130 μl of 100 mM tris-HCl buffer containing 1% triton, 10% glycerol and 2% anti-proteases, at pH 7.2, by carrying out three freezing at −80° C./thawing at 37° C. cycles, mechanical scraping of the cells, and then a sonication step.

The proteins present in the lysate are assayed using the Dc Protein assay kit (Biorad). The amount of glycosaminoglycans is measured in the supernatant and the lysate.

Assaying of Sulfated Glycosaminoglycans:

The glycosaminoglycans are assayed using the Blyscan glycosaminoglycan isolation & concentration kit (Biocolor, Newtownabbey, Northern Ireland) and the Blyscan sulfated glycosaminoglycan assay kit. The assay is performed on 700 μl and 400 μl of supernatant, 50 μl of lysate and a range of 0, 0.5, 1, 2, 3, 4 and 5 μg of supplied chondroitin-4-sulfate. Each sample to be measured is mixed with 700 μl of the cetylpyridinium chloride solution in a 1.5 ml centrifuge tube, and then incubated on a rotary mixer thermostated at 37° C., at 250 rpm, for 2 hours. The tubes are centrifuged for 15 min at 10,000 rpm in an Eppendorf 5415C centrifuge. The pellet is mixed with 1 ml of 95% ethanol and incubated in a rotary mixer thermostated at 37° C., at 250 rpm, for 5 min. The tube is again centrifuged for 15 min at 10,000 rpm. The ethanol is removed and the tube is dried in the open air for a few minutes. 1 ml of Blyscan dye reagent is added directly to the pellet and vortexed for 30 min at ambient temperature before being centrifuged at 14,000 rpm for 15 min. 1 ml of Blyscan dissociation reagent is added to the pellet. The whole is vortexed for 15 min until complete dissolution. The absorbance of the solution at 656 nm is measured using a polystyrene semi-microcuvette on a Biomate 5 spectrometer (Thermo). The amount of glycosaminoglycan present is then measured.

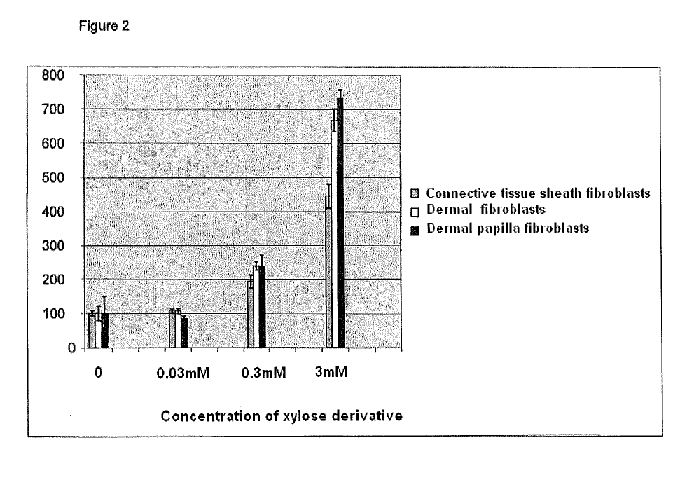

The results are represented in FIG. 2.

An increase in GAG synthesis by the fibroblasts, proportional to the concentration of 1,5-anhydro-6,8-dideoxy-L-glucooctitol, is observed. The addition of this active agent increases the amount of glycosaminoglycans secreted by up to 7 times.

### Example 3

### Hair-Straightening Composition

| | | |
|---|---|---|
| Ammonium polyacryloyldimethyltaurate | 0.5 | g |
| Porous particles of Nylon-12* | 4.7 | g |
| 5-n-octanoylsalicylic acid | 0.3 | g |
| Poloxamer 338 | 0.25 | g |
| Iduronosyl anhydromannitol | 1 | g |
| Demineralized water | 93.25 | g |

*The porous particles of Nylon-12 are marketed under the trademark “Orgasol 2002 UD Nat cos” by the company Atofina.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method of selecting/identifying an active agent for modifying the shape of keratin/hair fibers, which comprises the following steps:
a) culturing mammalian cells,
b) adding a test substance to the mammalian cell culture,
c) after incubation thereof, assaying the amount of glycosaminoglycans (GAGs) in the mammalian cells or in the mammalian cell culture medium,
d) comparing the amount of GAGs measured in step c) with that measured starting from a control culture of mammalian cells, obtained under the same conditions, but in the absence of test substance, and
e) selecting the substances for which the amount of GAGs is modified by at least a factor of 0.5 relative to the amount measured in the control culture.

2. The selection method as defined by claim 1, wherein the mammalian cells are fibroblasts or keratinocytes selected from among fibroblasts of the connective sheath of the hair, fibroblasts of the dermal papilla, fibroblasts of human dermis, skin keratinocytes, and keratinocytes of the outer sheath or of the epithelial matrix of the hair follicle.

3. The selection method as defined by claim 1, wherein, in step e), the substances for which the amount of GAGs is at least equal to 1.5 times the amount measured in the control culture are selected.

4. The selection method as defined by claim 1, wherein, in step e), the substances for which the amount of GAGs is decreased at least by half relative to the amount measured in the control culture are selected.

5. A regime or regimen for modifying the shape of keratin fibers, comprising administering to an individual in need of such treatment, at least one active agent selected by means of the method as defined by claim 1, wherein said at least one active agent is selected from among agents which stimulate the synthesis and/or secretion of GAGs and/or of PGs, and agents which decrease the degradation of GAGs.

6. The regime or regimen as defined by claim 5, wherein said at least one active agent comprises an agent for straightening and/or smoothing out keratin fibers.

7. A regime or regimen for modifying the shape of keratin fibers, comprising administering to an individual in need of such treatment, at least one active agent selected by means of the method as defined by claim 1, wherein said at least one active agent is selected from among agents which decrease or inhibit the synthesis and/or secretion of PGs and/or of GAGs, and/or which stimulate the degradation of GAGs.

8. The regime or regimen as defined by claim 7, wherein said at least one active agent comprises an agent for increasing the curve of keratin fibers.

9. The regime or regimen as defined by claim 5, wherein the keratin fibers are selected from human hair, body hair and/or eyelashes.

10. The regime or regimen as defined by claim 5, wherein said at least one agent for modifying the shape of keratin fibers comprises a modulator of the synthesis and/or secretion and/or degradation of GAGs, selected from among competitors of GAG synthesis, sugars which are GAG precursors, modulators of the enzymes which initiate GAG synthesis, modulators of the enzymes responsible for GAG synthesis elongation, modulators of the enzymes responsible for post-synthetic modifications of GAGs, heparanase 1 or 2 modulators, and glycanase modulators.

11. The regime or regimen as defined by claim 5, wherein said at least one agent for modifying the shape of keratin fibers is selected from among derivatives of iduronosyl anhydromannitol 6-sulfate (IMs), iduronosyl anhydromannitol, phenyl iduronide (Phl) or of 4-methylumbelliferyl iduronide, S-, N- and O-Glycosyl derivatives of the compound 2-acetamido-2-deoxy-D-glucose, or derivatives of 4-hydroxy-2-imino-1-methylpyrrolidine-5-carboxylic acid and of 2-imino-1-methylpyrrolidine-5-carboxylic acid, derivatives of aldonomonolactone or of alduronomonolactone, and acylated monosaccharides, and which compounds are active for straightening and/or smoothing out the hair.

12. The regime or regimen as defined by claim 5, wherein said at least one agent for modifying the shape of keratin fibers is selected from among analogues of 4-methylumbelliferone and of 1-deoxy-GlcNAc, 2-acetamido-3,4,6-tri-O-acetyl-1,5-anhydro-2-deoxy-D-glucitol, uridine derivatives, and 5′-(6-O-[10-(2-naphthyl)-3,6,9-trioxadecanyl]-alpha-d-galactopyranosyl)diphosphate, and which compounds are active for increasing the curve of the eyelash and/or for promoting or maintaining the curly and/or frizzy appearance of the hair, or for promoting the waviness of the hair.

13. The regime or regimen as defined by claim 5, comprising topically applying said at least one active agent onto said keratin fibers.

14. The regime or regimen as defined by claim 13, for caring for, conditioning, making up, removing makeup from, protecting, cleansing or washing keratin fibers.

15. The regime or regimen as defined by claim 5, wherein said at least one active agent for modifying the shape of keratin fibers comprises a composition which promotes its penetration into the hair follicle.

16. The regime or regimen as defined by claim 5, comprising co-administering at least one adjuvant selected from among silicones in soluble, dispersed or microdispersed form, nonionic, anionic, cationic and amphoteric surfactants, ceramides, glycoceramides and pseudoceramides, vitamins and pro-vitamins, panthenol, plant, animal, mineral and synthetic oils, waxes other than the ceramides, glycoceramides and pseudoceramides, silicone-based or nonsilicone-based, water-soluble and fat-soluble sunscreens, pearlescent agents and opacifiers, sequestering agents, plasticizers, solubilizing agents, acidifying agents, mineral and organic thickeners, antioxidants, hydroxy acids, penetration agents, fragrances and preservatives.

17. The regime or regimen as defined by claim 5, said at least one active agent comprising a cosmetic composition.

18. A cosmetic treatment regime or regimen for modifying the shape of keratin fibers in a mammal, wherein at least one active agent which modulates the synthesis, secretion and/or degradation of GAGs, or composition comprised thereof, or selected by means of the method as defined by claim 1, is applied onto the keratin fibers and/or onto the support thereof.

19. A composition useful for modifying the shape of keratin/hair fibers, comprising a thus effective amount of at least one active agent selected from among those which stimulate the synthesis and/or secretion of GAGs and/or of PGs, and agents which decrease the degradation of GAGs, formulated into a physiologically acceptable medium therefor.

20. The composition as defined by claim 19, comprising at least one active agent for straightening and/or smoothing our keratin fibers.

21. A composition useful for modifying the shape of keratin/hair fibers, comprising a thus effective amount of at least one active agent selected from among those which decrease or inhibit synthesis and/or secretion of PGs and/or of GAGs, and/or which stimulate the degradation of GAGs, formulated into a physiologically acceptable medium therefor.

22. The composition as defined by claim 21, comprising at least one active agent for increasing the curve of keratin fibers.

23. A composition useful for modifying the shape of keratin/hair fibers, comprising a thus effective amount of at least one active agent selected from among those which modulate the synthesis and/or secretion and/or degradation of GAGs, selected from among competitors of GAG synthesis, sugars which are GAG precursors, modulators of the enzymes which initiate GAG synthesis, modulators of the enzymes responsible for GAG synthesis elongation, modulators of the enzymes responsible for post-synthetic modifications of GAGs, heparanase 1 or 2 modulators, and glycanase modulators, formulated into a physiologically acceptable medium therefor.

24. A composition useful for straightening and/or smoothing out the hair, comprising a thus effective amount of at least one active agent selected from among derivatives of iduronosyl anhydromannitol 6-sulfate (IMs), iduronosyl anhydromannitol, phenyl iduronide (Phl) or of 4-methylumbelliferyl iduronide, S-, N- and O-Glycosyl derivatives of the compound 2-acetamido-2-deoxy-D-glucose, or derivatives of 4-hydroxy-2-imino-1-methylpyrrolidine-5-carboxylic acid and of 2-imino-1-methylpyrrolidine-5-carboxylic acid, derivatives of aldonomonolactone or of alduronomonolactone, and acylated monosaccharides, formulated into a physiologically acceptable medium therefor.

25. A composition useful for increasing the curve of the eyelash and/or for promoting or maintaining the curly and/or frizzy appearance of the hair, or for promoting the waviness of the hair, comprising a thus effective amount of at least one active agent selected from among analogues of 4-methylumbelliferone and of 1-deoxy-GlcNAc, 2-acetamido-3,4,6-tri-O-acetyl-1,5-anhydro-2-deoxy-D-glucitol, uridine derivatives, and 5′-(6-O-[10-(2-naphthyl)-3,6,9-trioxadecanyl]-alpha-d-galactopyranosyl)diphosphate, formulated into a physiologically acceptable medium therefor.
